(19) United States
(12) Patent Application Publication
(10) Pub. No.: US 2021/0298627 A1
(43) Pub. Date: Sep. 30, 2021

(54) EEG SIGNAL GENERATION NETWORK, METHOD AND STORAGE MEDIUM

(71) Applicant: Wuyi University, Jiangmen (CN)

(72) Inventors: Hongtao WANG, Jiangmen (CN); Cong TANG, Jiangmen (CN); Zi'an PEI, Jiangmen (CN); Linfeng XU, Jiangmen (CN)

(21) Appl. No.: 17/004,822

(22) Filed: Aug. 27, 2020

(30) Foreign Application Priority Data
Mar. 26, 2020 (CN) 2020102215357

(51) Int. Cl. A61B 5/04; A61B 5/0476; A61B 5/00

(52) U.S. Cl. CPC A61B 5/04012; A61B 5/0476; G06N 20/00; A61B 5/7267; A61B 5/7221; A61B 5/04004

(57) ABSTRACT

Disclosed are an EEG signal generation network, method and storage medium. The EEG signal generation network includes a real EEG signal input end, a real EEG signal labeling module, a generator, a sharing module, a discriminator, and a classifier. The EEG signal generation network is configured to minimize losses of the generator, discriminator and classifier, and to minimize a combined loss of the discriminator and classifier through training, and to generate a new event-related potential.

### CROSS-REFERENCE TO RELATED APPLICATIONS

This application is based on and claims the benefit of priority from Chinese Patent Application No. 2020102215357 filed on 26 Mar. 2020, the entirety of which is incorporated by reference herein.

### TECHNICAL FIELD

The present disclosure relates to the field of biological information technology, and more particularly, to an EEG signal generation network, method and storage medium.

### BACKGROUND

EEG signals are the overall reflection of the electrophysiological activities of brain nerve cells on the surface of the cerebral cortex or scalp. In engineering applications, EEG signals are used to realize the brain-computer interface, and the difference in EEG signals generated by different senses, movements, or cognitive activities is used to analyze and process the EEG signals. When applied to research, a large amount of high-quality EEG signal data is required, but it takes time, manpower, and material resources to acquire a large amount of high-quality EEG signal. Event-related potentials are a special kind of brain evoked potentials, which are brain potentials caused by multiple or multiple intentionally given stimuli. It reflects the neuroelectrophysiological changes of the brain during the cognitive process. EEG signals can be studied more quickly through event-related potentials. However, the current EEG signal generation network is generally affected by training instability and mode collapse. It can only generate low-resolution samples, and cannot effectively classify the samples to event-related potentials.

### SUMMARY

The aim of the present disclosure is to solve at least one of the technical problems existing in the prior art, by providing an EEG signal generation network, and a method and storage medium thereof.

According to a first aspect of the present disclosure, an EEG signal generation network comprises:

a real EEG signal input end, configured to input a real EEG signal comprising an event-related potential and a non-event-related potential;

a real EEG signal labeling module, configured to generate a real sample by combining the real EEG signal with a real classification label comprising a first label labeling the event-related potential and a second label labeling the non-event-related potential;

a generator, configured to generate a multi-channel reconstructed sample by combining a noise signal with a randomly generated classification label, the generator provided with an upsampling layer comprising a convolutional layer with bicubic interpolation and a deconvolutional layer with bilinear weight initialization, the randomly generated classification label comprising the first label labeling the event-related potential and the second label labeling the non-event-related potential;

a sharing module, configured to combine the real sample and the reconstructed sample into a total sample, and to distribute an output;

a discriminator, configured to determine that each data in the total sample is the real EEG signal or the noise signal, the discriminator having a gradient loss function based on Wasserstein distance, and the discriminator and the generator forming an adversarial relationship; and

a classifier, configured to classify each data in the total sample as the event-related potential or the non-event-related potential, and to determine a correctness of classification result according to a total classification label comprising the real classification label and the randomly generated classification label;

wherein the EEG signal generation network is configured to, through training, minimize losses of the generator, the discriminator and the classifier, and to minimize a combined loss of the discriminator and the classifier, and to generate a new event-related potential.

According to the first aspect of the present disclosure, the loss of the discriminator is as follows:

L_{D}(ϕ_{G}*, ϕ_{D}, ϕ_{H})=\tilde{W}(T_{y}, T_{f});

the loss of the classifier is as follows: L_{C}(ϕ_{G}*, ϕ_{C}, ϕ_{H})=E[log T(y [symbol] |X [symbol] )]+E└log T(y_{f}|X_{f})┘; the loss of the generator is as follows: L_{G}(ϕ_{G}, ϕ_{D}*, ϕ_{C}*, ϕ_{H}*)=E [symbol] └D_{ϕ}_{_{D}}′(G_{ϕ}_{_{G}}(z,y_{f}))┘+L_{C}(ϕ_{G}, ϕ_{C}*, ϕ_{H}*); the combined loss is as follows: L_{D/C}(ϕ_{G}*, ϕ_{D}, ϕ_{C}, ϕ_{H}*)=L_{D}(ϕ_{G}*, ϕ_{D}, ϕ_{H}*)−L_{C}(ϕ_{G}*, ϕ_{C}*, ϕ_{H}).

According to the first aspect of the present disclosure, wherein the generator comprises: a first input layer, a first fully connected layer, a first ReLU function, a second fully connected layer, a first normalization function, a second ReLU function, the upsampling layer, a cropping layer, a second normalization function, a third ReLU function, a first convolution layer and a first output layer connected in sequence.

According to the first aspect of the present disclosure, wherein the generator inputs the noise signal generated by a multi-dimensional standard normal distribution through the first input layer; the first input layer is also used to add the randomly generated classification label.

According to the first aspect of The present disclosure, wherein the discriminator adopts a CNN architecture; the discriminator comprises a second input layer, a second convolution layer, a fourth ReLU function, a third convolution layer, a fifth ReLU function, a fourth convolution layer, a third fully connected layer, a fourth fully connected layer, a sixth ReLU function, a fifth fully connected layer and a second output layer connected in sequence.

According to the first aspect of the present disclosure, the discriminator adds Gaussian white noise to the total sample before the second convolutional layer to avoid zero gradient.

According to a second aspect of the present disclosure, a method for generating EEG signal comprises:

collecting real EEG signals;

preprocessing the real EEG signals;

the preprocessed real EEG signals are input to the EEG signal generation network described above to generate a new event-related potential.

According to the second aspect of the present disclosure, collecting real EEG signals comprises:

collecting EEG signals generated when multiple subjects view a character matrix through an EEG signal collection instrument, wherein a plurality of characters within the character matrix is flashed randomly at a rated frequency;

the event-related potential is a potential signal generated by the subject seeing a flashing of a specified character, and the non-event-related potential is a potential signal generated by the subject seeing a flashing of a plurality of characters that do not comprise the specified character.

According to the second aspect of the present disclosure, preprocessing the real EEG signals comprises:

performing low-pass filtering on the real EEG signals; aligning waveforms of the multiple real EEG signals according to a time axis, and taking an average value after accumulation.

According to a third aspect of the present disclosure, a storage medium storing executable instructions, the executable instructions are executed by a computer to cause the computer to execute the method for generating EEG signal according to the first aspect of the present disclosure.

The above solution has at least the following beneficial effects: the generator contains a convolutional layer with bicubic interpolation and an upsampling layer with a deconvolution layer with bilinear weight initialization, so that the reconstructed samples generated by the generator can achieve the desired efficiency of deceiving the discriminator; by setting classification label and adding classifier, the generation rate of event-related potentials is increased, and the application of the generative adversarial networks in the field of brain-computer interface and the application of classification are realized; using Wasserstein distance to effectively improve the stability and convergence of training; the EEG signal generation network can efficiently generate a large amount of high-quality event-related potential data.

Additional aspects and advantages of the present disclosure will be partially given in the following description, and some will become apparent from the following description, or be learned through the practice of the present disclosure.

### BRIEF DESCRIPTION OF THE DRAWINGS

The present disclosure will be further described below with reference to the drawings and examples.

FIG. 1 is a schematic diagram of an EEG signal generation network according to an embodiment of the present disclosure;

FIG. 2 is a network structure diagram of a generator;

FIG. 3 is a network structure diagram of a discriminator;

FIG. 4 is a bar graph of the recognition accuracy of the EEG signal generation network on the event-related potentials by taking 5 times accumulated EEG signal as input;

FIG. 5 is a bar graph of the recognition accuracy of the EEG signal generation network on the event-related potentials by taking 10 times accumulated EEG signal as input;

FIG. 6 is a diagram of the effect detection of the EEG signal generation network on the event-related potentials by taking 5 times accumulated EEG signal as input;

FIG. 7 a diagram of the effect detection of the EEG signal generation network on the non-event-related potentials by taking 5 times accumulated EEG signal as input;

FIG. 8 is a diagram of the effect detection of the EEG signal generation network on the event-related potentials by taking 10 times accumulated EEG signal as input;

FIG. 9 is a diagram of the effect detection of the EEG signal generation network on the non-event-related potentials by taking 10 times accumulated EEG signal as input.

### DETAILED DESCRIPTION

Specific embodiments of the present disclosure will be described in detail in this section. Preferred embodiments of the present disclosure are shown in the accompanying drawings which function to supplement the description of the written description with graphics, so that each technical feature and the overall technical solution of the present disclosure can be intuitively and vividly understood, but it cannot be construed as limiting the protection scope of the present disclosure.

In the description of the present disclosure, “several” means one or more, and “a plurality of” means more than two, “greater than, less than, more than, etc.,” are understood as not including the number itself, while “above, below, within, etc.,” are understood as including the number itself. It should be noted that the terms first and second are only used to distinguish technical features, and cannot be understood as indicating or implying relative importance or implicitly indicating the number of technical features indicated or implicitly indicating the precedence of the technical features indicated.

In the description of the present disclosure, unless otherwise clearly defined, the terms such as “arrange”, “install” and “connect” shall be understood in a broad sense. A person skilled in the art can reasonably determine the specific meanings of the above terms in the present disclosure in combination with specific contents of the technical solution.

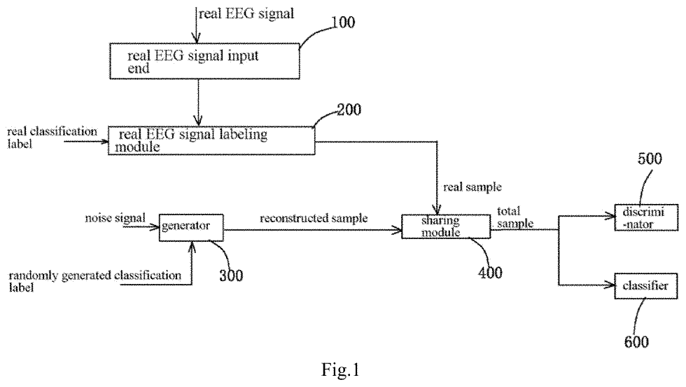

Referring to FIG. 1, according to an embodiment of the present disclosure, an EEG signal generation network includes:

a real EEG signal input end 100 for inputting a real EEG signal comprising an event-related potential and a non-event-related potential;

a real EEG signal labeling module 200 for combining a real EEG signal with a real classification label to generate a real sample, the real classification label comprises a first label labeling the event-related potential and a second label labeling the non-event-related potential;

a generator 300 for combining a noise signal with a randomly generated classification label to generate a multi-channel reconstructed sample, the generator is provided with an upsampling layer 34 comprising a convolutional layer 341 with bicubic interpolation and a deconvolutional layer 342 with bilinear weight initialization, the randomly generated classification label includes the first label labeling the event-related potential and the second label labeling the non-event-related potential;

a sharing module 400 for combining the real sample and the reconstructed sample into a total sample and distributing an output;

a discriminator 500 for determining that each data in the total sample is the real EEG signal or the noise signal, the discriminator 500 has a gradient loss function based on Wasserstein distance, and the discriminator 500 and the generator 300 form an adversarial relationship;

a classifier 600 for classifying each data in the total sample as the event-related potential or the non-event-related potential, and determining a correctness of classification result according to a total classification label, the total classification label comprises the real classification label and the randomly generated classification label;

through training, losses of the generator 300, the discriminator 500, and the classifier 600 are minimized and a combined loss of the discriminator 500 and the classifier 600 is minimized, and a new event-related potential is generated.

In this embodiment, the real EEG signal is input through the real EEG signal input end 100, and the real EEG signal includes event-related potentials and non-event-related potentials. The real EEG signal labeling module 200 labels the first label as an event-related potential and the second label as a non-event-related potential, then the real sample is actually composed of the event-related potential labeled with the first label and the non-event-related potential labeled with the second label.

Referring to FIG. 2, for the generator 300, a noise signal is randomly generated by an external signal generation module from a 300-dimensional standard normal distribution and input to the generator 300. The noise signal is input from a first input layer 31; a randomly generated classification label is added to the noise signal at the first input layer 31, and the classification label added to the noise signal includes the first label and the second label. The noise signal passes through a first fully connected layer 32, a first ReLU function, a second fully connected layer 33, a first normalization function, a second ReLU function, a upsampling layer 34, a cropping layer 35, a second normalization function, a third ReLU function, a first convolution layer 36 to generate 32-channel reconstructed samples. The reconstructed samples are output to the sharing module 400 through the first output layer 37. The reconstructed sample also includes the event-related potential labeled with the first label and the non-event-related potential labeled with the second label.

Specifically, the first fully connected layer 32 has 1024 neurons, and the second fully connected layer 33 has 73728 neurons; the first ReLU function, the second ReLU function, and the third ReLU function are all Leaky Relu functions. After activation by the second ReLU function, the size of the signal entering the upsampling layer 34 is 9×64×128. In the upsampling layer 34, with a factor of 2 times, the size of the signal is increased to 18×128×128 after a first upsampling. The first upsampling is performed in the convolutional layer 341 with double cubic interpolation, after a second upsampling, the size of the signal is increased to 36×256×128. The second upsampling is performed on the deconvolution layer 342 with bilinear weight initialization. Cropping into a signal of size 32×160×128 through the cropping layer 35, generating a signal of size 32×160×1 after passing through the second normalization function and the third ReLU function, using a convolutional layer with a 3×3 kernel to generate 32 channels of reconstructed samples, the reconstructed sample is actually a two-dimensional EEG signal image.

It should be noted that different up-sampling layers 34 will have different effects on the frequency and amplitude of EEG signals. The upsampling combination includes DC-DC performing two deconvolutions, EEG-GAN-BCBC performing two bicubic interpolations, EEG-GAN-NNNN performing two nearest neighbor interpolations, and deconvoluted DCBL-DCBL with two bilinear weight initializations. However, DC-DC and DCBL-DCBL will produce relatively low amplitude artifacts, which is mainly due to the “checkerboard effect” of deconvolution; on the other hand, EEG-GAN-BCBC and EEG-GAN-NNNN can match the frequency of the signal, but cannot generate the correct amplitude. Compared with the above upsampling method, the use of the upsampling layer 34 is more conducive to the generator 300 to generate reconstructed samples, so that the reconstructed samples generated by the generator 300 can reach the expected efficiency of deceiving the discriminator 500, which also provides better performance for reducing artifacts and improving network training and classification.

In the sharing module 400, the real samples and the reconstructed samples are combined into a total sample, which is then distributed to the classifier 600 and the discriminator 500. The sharing module 400 is provided with a sharing layer which is used to distribute and output the total samples. It should be noted that the step of combining real samples and reconstructed samples into a total sample is done outside the shared layer. The classifier 600 and the discriminator 500 jointly use the total samples in the sharing module 400.

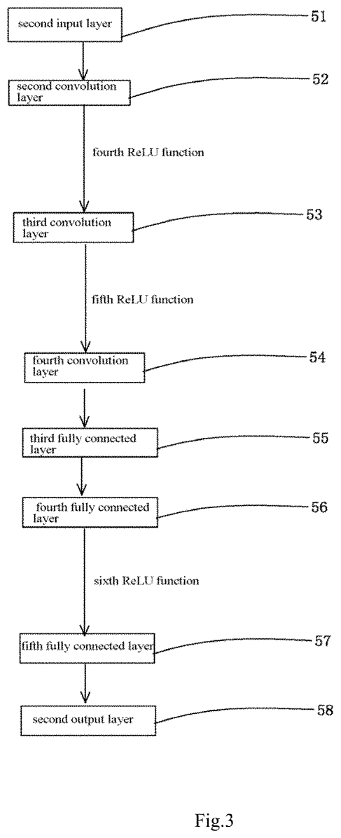

Referring to FIG. 3, the discriminator 500 adopts a CNN architecture; the discriminator 500 includes a second input layer 51, a second convolution layer 52, a fourth ReLU function, a third convolution layer 53, a fifth ReLU function, a fourth convolution layer 54, and a third fully connected layer 55, a fourth fully connected layer 56, a sixth ReLU function, a fifth fully connected layer 57 and a second output layer 58 that are sequentially connected. Specifically, the size of the signal entering the second convolution layer 52 is 32×160×64, and the size of the signal entering the third convolution layer 53 after being processed by the fourth ReLU function is 32×80×128, the size of the signal processed by the fifth ReLU function and entering the fourth convolution layer 54 is 8×40×128; the third fully connected layer 55 has 40960 neurons, the fourth fully connected layer 56 has 1024 neurons, and the fifth fully connected layer 57 has 1 neuron.

In addition, the discriminator 500 adds Gaussian white noise with an average value of 0 and a standard deviation of 0.05 to the total sample before the second convolution layer 52 to avoid zero gradient and improve the training stability of the discriminator 500. The size of the signal entering the second convolution layer 52 is 32×160×64.

Since the discriminator 500 and the generator 300 are adversarial and competing network modules, the discriminator 500 needs to determine whether each data in the total sample is a real EEG signal or a noise signal, that is, whether the data is real or reconstructed. The task of the generator 300 is to generate a “real” reconstructed sample to deceive the discriminator 500. This can easily lead to minimax decision, which can make the network unstable. The problem is solved by Wasserstein distance, which is calculated according to the following formula:

W̅(T_{y}, T_{f})=Eₓ [symbol] _{└}D_{ϕ}_{_{D}}(X_{y})┘−Eₓ [symbol] └D_{ϕ}_{_{D}}(X_{f})┘;

Xᵣ represents the real sample, X_{f} represents the reconstructed sample, Tᵣ represents the distribution of the real sample, T_{f} represents the distribution of the reconstructed sample; φ_{D} represents the parameter that determines the loss of the discriminator 500. In addition, using the Wasserstein distance requires the discriminator 500 to have K-Lipschitz continuity, it is necessary to cut the weight of the discriminator 500D into the interval [−c, c] to achieve this purpose. At the same time, in order to better achieve K-Lipschitz continuity on the discriminator 500, which is achieved by adding a gradient loss function to the loss of the EEG signal generation network, the gradient loss function is as follows:

\tilde{W}(T_{y}, T_{f})=\tilde{W}(T_{y}, T_{f})+λE_{\hat{X}} [symbol] _{T}_{_{\hat{X}└(∥∇}}_{\hat{X}}D(\hat{X})∥₂−1)²┘,

where λ is the hyperparameter that controls the weigh between the loss of the EEG signal generation network and the gradient loss function, \hat{X} indicating that the total sample lies on a straight line between Tᵣ and T_{f}.

By training the discriminator 500, the Wasserstein distance can be minimized, that is, the loss L_{D}(ϕ_{D}, ϕ_{G}*)=W̅(T_{y}, T_{f}) of the discriminator 500 can be reduced, thus the stability and convergence of the training are effectively improved, and the generation of high-resolution samples is facilitated. φ_{G} represents a parameter that determines the loss of the generator 300. The parameter with * indicates that the parameter has been determined as a fixed value.

For the classifier 600 identifies each data of the total sample to generate an identification label, and then checks the total classification label of each data to confirm whether the classification result of the classifier 600 is correct. The classifier 600 feeds back information to the generator 300 according to the accuracy and loss of the classification result. The classification label is used for supervised learning, and plays a role in optimizing the generated reconstructed samples, which is helpful for the generator 300 to generate event-related potentials. In the overall training process of the EEG signal generation network, for a fixed φ_{G}, the loss of the classifier 600 is minimized. The loss of the classifier 600 is as follows:

L_{C}(ϕ_{G}*, ϕ_{C}, ϕ_{H})=E[log T(y_{y}|X_{y})]+E└log T(y_{f}|X_{f})┘.

y_{f} is the label of the event-related potential. φ_{H} represents a parameter that determines the loss of the shared module 400.

In addition, through training, for a fixed φ_{G}, the combined loss of the discriminator 500 and the classifier 600 is reduced in a maximum extent, and the combined loss is as follows:

L_{D/C}(ϕ_{G}*, ϕ_{D}, ϕ_{C}, ϕ_{H}*)=L_{D}(ϕ_{G}*, ϕ_{D}, ϕ_{H}*)−L_{C}(ϕ_{G}*, ϕ_{C}, ϕ_{H}).

Finally, the correction loss of the generator 300 is minimized. Meanwhile, φ_{D}, φ_{C} and φ_{H} H are fixed values, and the correction loss of the generator 300 is

L_{G}(ϕ_{G}, ϕ_{D}*, ϕ_{C}*, ϕ_{H}*)=Eₓ [symbol] _{└}D [symbol] _{(G} [symbol] (z,y_{f}))┘+L_{C}(ϕ_{G}, ϕ_{C}*, ϕ_{H}*).

At this time, the reconstructed samples generated by the generator 300 are optimal, the discriminator 500 cannot discriminate the authenticity of the reconstructed samples generated by the generator 300, and most of the reconstructed samples are event-related potentials.

When the losses of the generator, discriminator, and classifier are minimized and the combined losses of the discriminator and classifier are minimized, the EEG signal generation network achieve global convergence.

The EEG signal generation network can efficiently generate a large amount of high-quality event-related potential data, which solves the problem of small data samples in the field of brain-computer interface.

According to another embodiment of the present disclosure, a method for generating EEG signals includes the following steps:

collecting real EEG signals;

preprocessing the real EEG signals;

the preprocessed real EEG signals are input to the EEG signal generation network described above to generate a new event-related potential.

In this method embodiment, since the same EEG signal generation network as described above is used to generate a new time-related potential, accordingly, the processing steps of the EEG signal generation network are as above, and will not be described in detail here. Similarly, it has the same beneficial effects.

Further, collecting real EEG signals comprises: collecting EEG signals generated when multiple subjects view a character matrix through an EEG signal collection instrument, wherein a plurality of characters within the character matrix is flashed randomly at a rated frequency; the event-related potential is a potential signal generated by the subject seeing a flashing of a specified character, and the non-event-related potential is a potential signal generated by the subject seeing a flashing of a plurality of characters that do not comprise the specified character.

26 English alphabet characters, 9 numeric characters and one symbol character form a 6×6 character matrix. single row or single column characters in the character matrix are continuously and randomly flashed at a frequency of 5.7 Hz. The ratio of event-related potentials to non-event-related potentials in the collected real EEG signals is preferably 1:5. The designated character is a character or characters in the character matrix designated by the operator.

Further, preprocessing the real EEG signal is specifically: low-pass filtering the real EEG signal with a cut-off frequency of 20 Hz, to retain the real EEG signal with a frequency concentrated between 0.1-20 Hz, and to remove noise signal components in unrelated frequency bands; aligning the waveforms of multiple real EEG signals along the time axis, and obtaining an average value after accumulation. In order to obtain the event-related potential completely, a size of a time window is preferably 0 ms-667 ms, and a size of the resulting data is 32×160.

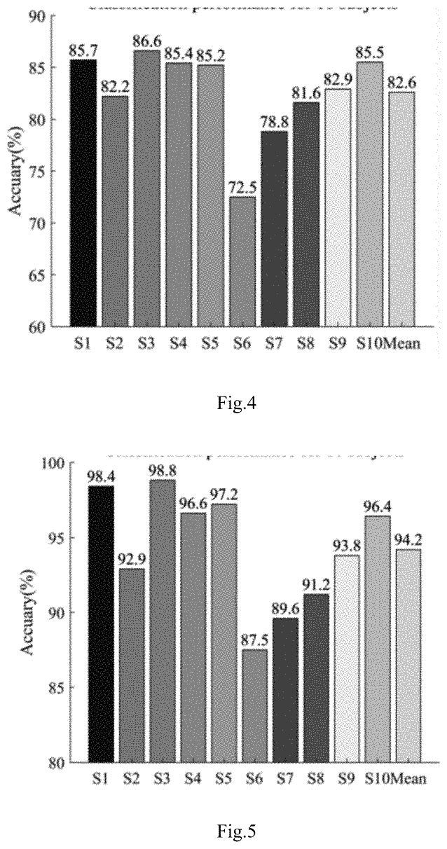

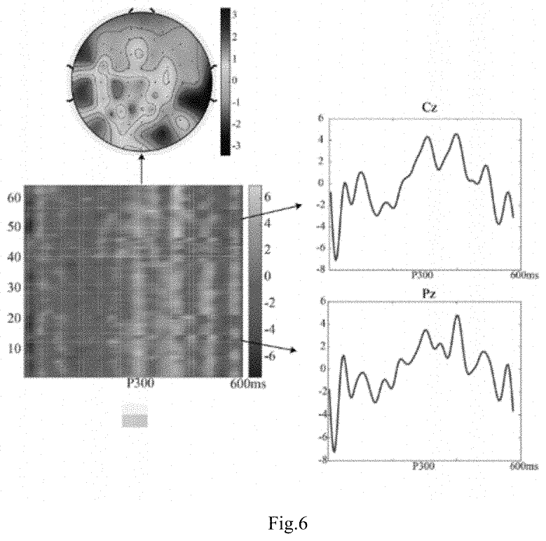

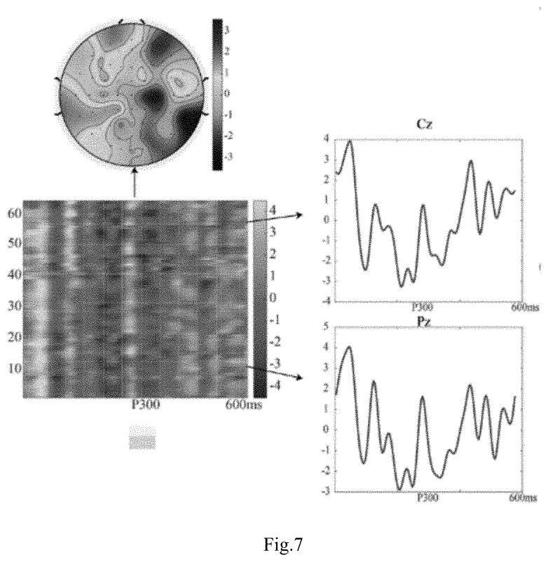

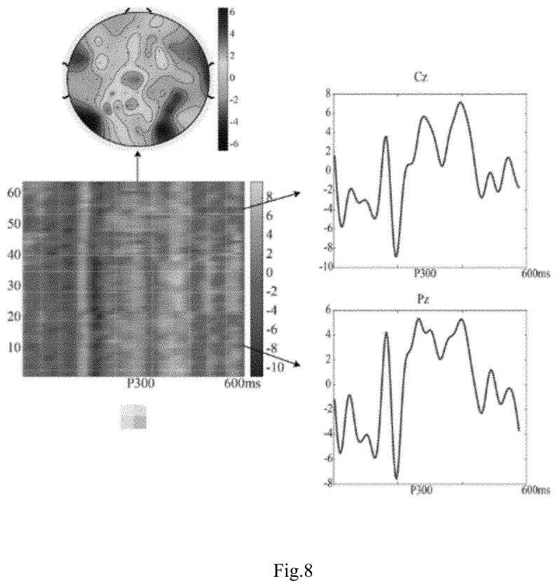

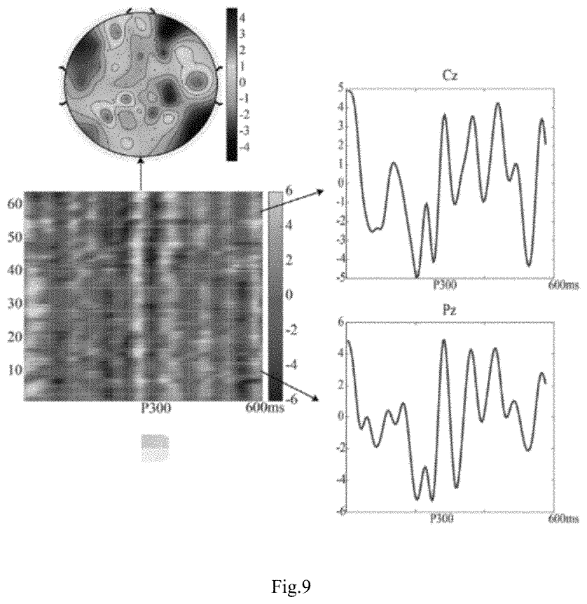

Specifically, in the experiment, the waveforms of multiple real EEG signals are aligned according to the time axis and are accumulated 5 times, and then averaged; and the waveforms of multiple real EEG signals are aligned along the time axis and are accumulated 10 times, and then averaged. Then these two pre-processed results are input into the EEG signal generation network. Examining the classification effect of the EEG signal generation network, the results are shown in FIGS. 4 and 5, and it can be seen that, the EEG signal generation network has a high accuracy of event-related potential recognition and has an excellent classification effect. Examining the quality of event-related potentials generated by the EEG signal generation network, the results are shown in FIGS. 6 to 9, it can be seen that the quality of the event-related potentials in the reconstructed samples generated by the EEG signal generation network is high, which can achieve the effect of being close to the event-related potentials of real EEG signals.

According to another embodiment of the present disclosure, a storage medium storing executable instructions is provided, the executable instructions are executed by a computer to cause the computer to execute the method for generating EEG signal according to the first aspect of The present disclosure.

Examples of storage medium include, but are not limited to phase change memory (PRAM), static random access memory (SRAM), dynamic random access memory (DRAM), other types of random access memory (RAM), read only memory (ROM), electrically erasable programmable read-only memory (EEPROM), flash memory or other memory technology, CD-ROM (CD-ROM), digital versatile disc (DVD) or other optical storage, magnetic cassette tape, magnetic tape magnetic disk storage or other magnetic storage devices or any other non-transmission media, which can be used to store information that can be accessed by a computing device.

The above are only preferred examples of the present disclosure, and the present disclosure is not limited to the above-mentioned embodiments, as long as they achieve the technical effects of the present disclosure by the same means, they should fall within the protection scope of the present disclosure.

We claim:

1. an EEG signal generation network, comprising:
a real EEG signal input end, configured to input a real EEG signal comprising an event-related potential and a non-event-related potential;
a real EEG signal labeling module, configured to generate a real sample by combining the real EEG signal with a real classification label comprising a first label labeling the event-related potential and a second label labeling the non-event-related potential;
a generator, configured to generate a multi-channel reconstructed sample by combining a noise signal with a randomly generated classification label, the generator provided with an upsampling layer comprising a convolutional layer with bicubic interpolation and a deconvolutional layer with bilinear weight initialization, the randomly generated classification label comprising the first label labeling the event-related potential and the second label labeling the non-event-related potential;
a sharing module, configured to combine the real sample and the reconstructed sample into a total sample, and to distribute an output;
a discriminator, configured to determine that each data in the total sample is the real EEG signal or the noise signal, the discriminator having a gradient loss function based on Wasserstein distance, and the discriminator and the generator forming an adversarial relationship; and
a classifier, configured to classify each data in the total sample as the event-related potential or the non-event-related potential, and to determine a correctness of classification result according to a total classification label comprising the real classification label and the randomly generated classification label;
wherein the EEG signal generation network is configured to, through training, minimize losses of the generator, the discriminator and the classifier, and to minimize a combined loss of the discriminator and the classifier, and to generate a new event-related potential.

2. The network of claim 1, wherein the loss of the discriminator is as follows:
L_{D}(ϕ_{G}*, ϕ_{D}, ϕ_{H})=\tilde{W}(T_{y}, T_{f});
the loss of the classifier is as follows:
L_{C}(ϕ_{G}*, ϕ_{C}, ϕ_{H})=E[log T(y_{y}|X_{y})]+E└log T(y_{f}|X_{f})┘;
the loss of the generator is as follows:
L_{G}(ϕ_{G}, ϕ_{D}*, ϕ_{C}*, ϕ_{H}*)=Eₓ [symbol] └D [symbol] _{(G} [symbol] (z,y_{f}))┘+L_{C}(ϕ_{G}, ϕ_{C}*, ϕ_{H}*);
the combined loss is as follows:
L_{D/C}(ϕ_{G}*, ϕ_{D}, ϕ_{C}, ϕ_{H}*)=L_{D}(ϕ_{G}*, ϕ_{D}, ϕ_{H}*)−L_{C}(ϕ_{G}*, ϕ_{C}, ϕ_{H}).

3. The network of claim 2, wherein the generator comprises:
a first input layer, a first fully connected layer, a first ReLU function, a second fully connected layer, a first normalization function, a second ReLU function, the upsampling layer, a cropping layer, a second normalization function, a third ReLU function, a first convolution layer and a first output layer connected in sequence.

4. The network of claim 3, wherein the generator receives the noise signal generated by a multi-dimensional standard normal distribution through the first input layer, and the first input layer is further configured to add the randomly generated classification label.

5. The network of claim 2, wherein the discriminator adopts a CNN architecture, and comprises a second input layer, a second convolution layer, a fourth ReLU function, a third convolution layer, a fifth ReLU function, a fourth convolution layer, a third fully connected layer, a fourth fully connected layer, a sixth ReLU function, a fifth fully connected layer and a second output layer connected in sequence.

6. The network of claim 5, wherein the discriminator is configured to adds Gaussian white noise into the total sample before the second convolutional layer to avoid zero gradient.

7. A method for generating EEG signal, comprising:
collecting real EEG signals;
preprocessing the real EEG signals to obtain preprocessed EEG signals;
inputting the preprocessed real EEG signals into an EEG signal generation network to generate a new event-related potential, the EEG signal generation network comprising:
a real EEG signal input end, configured to input a real EEG signal comprising an event-related potential and a non-event-related potential;
a real EEG signal labeling module, configured to generate a real sample by combining the real EEG signal with a real classification label comprising a first label labeling the event-related potential and a second label labeling the non-event-related potential;
a generator, configured to generate a multi-channel reconstructed sample by combining a noise signal with a randomly generated classification label, the generator provided with an upsampling layer comprising a convolutional layer with bicubic interpolation and a deconvolutional layer with bilinear weight initialization, the randomly generated classification label comprising the first label labeling the event-related potential and the second label labeling the non-event-related potential;
a sharing module, configured to combine the real sample and the reconstructed sample into a total sample, and to distribute an output;
a discriminator, configured to determine that each data in the total sample is the real EEG signal or the noise signal, the discriminator having a gradient loss function based on Wasserstein distance, and the discriminator and the generator forming an adversarial relationship; and
a classifier, configured to classify each data in the total sample as the event-related potential or the non-event-related potential, and to determine a correctness of classification result according to a total classification label comprising the real classification label and the randomly generated classification label;
wherein the EEG signal generation network is configured to, through training, minimize losses of the generator, the discriminator and the classifier, and to minimize a combined loss of the discriminator and the classifier, and to generate a new event-related potential.

8. The method of claim 7, wherein collecting real EEG signals comprises:
collecting EEG signals generated when multiple subjects view a character matrix through an EEG signal collection instrument, wherein a plurality of characters within the character matrix is flashed randomly at a rated frequency;
the event-related potential is a potential signal generated by the subject seeing a flashing of a specified character, and the non-event-related potential is a potential signal generated by the subject seeing a flashing of a plurality of characters that do not comprise the specified character.

9. The method of claim 7, wherein preprocessing the real EEG signals comprises:
performing low-pass filtering on the real EEG signals;
aligning waveforms of the multiple real EEG signals according to a time axis, and taking an average value after accumulation.

10. A storage medium storing executable instructions, the executable instructions are executed by a computer to cause the computer to execute a method for generating EEG signal, the method comprising:
collecting real EEG signals;
preprocessing the real EEG signals to obtain preprocessed EEG signals;
inputting the preprocessed real EEG signals into an EEG signal generation network to generate a new event-related potential, the EEG signal generation network comprising:
a real EEG signal input end, configured to input a real EEG signal comprising an event-related potential and a non-event-related potential;
a real EEG signal labeling module, configured to generate a real sample by combining the real EEG signal with a real classification label comprising a first label labeling the event-related potential and a second label labeling the non-event-related potential;
a generator, configured to generate a multi-channel reconstructed sample by combining a noise signal with a randomly generated classification label, the generator provided with an upsampling layer comprising a convolutional layer with bicubic interpolation and a deconvolutional layer with bilinear weight initialization, the randomly generated classification label comprising the first label labeling the event-related potential and the second label labeling the non-event-related potential;
a sharing module, configured to combine the real sample and the reconstructed sample into a total sample, and to distribute an output;
a discriminator, configured to determine that each data in the total sample is the real EEG signal or the noise signal, the discriminator having a gradient loss function based on Wasserstein distance, and the discriminator and the generator forming an adversarial relationship; and
a classifier, configured to classify each data in the total sample as the event-related potential or the non-event-related potential, and to determine a correctness of classification result according to a total classification label comprising the real classification label and the randomly generated classification label;
wherein the EEG signal generation network is configured to, through training, minimize losses of the generator, the discriminator and the classifier, and to minimize a combined loss of the discriminator and the classifier, and to generate a new event-related potential.
